(19) United States
(12) Patent Application Publication
(10) Pub. No.: US 2009/0163807 A1
(43) Pub. Date: Jun. 25, 2009

(54) FINGER-MOUNTED OR ROBOT-MOUNTED TRANSDUCER DEVICE

(76) Inventor: John W. Sliwa, Los Altos Hills, CA (US)

Correspondence Address: SJM/AFD - DYKEMA;c/o CPA Global, P.O. Box 52050, Minneapolis, MN, 55402, US

(21) Appl. No.: 11/962,850

(22) Filed: Dec. 21, 2007

(51) Int. Cl. A61B 8/00

(52) U.S. Cl. 600/439

(57) ABSTRACT

A transducer device for therapeutic applications is disclosed. The transducer device may include a mounting body configured for mounting the device to a finger of an operator of the device. The transducer device may also include a transducer housing connected to the mounting body that defines a receiving portion. The transducer device may further include a transducer element disposed in the receiving portion that is configured for connection to an energy supply and configured to transmit energy from an emitting surface. The transducer device may further include a gas reservoir disposed between the transducer element and the mounting body that is configured to prevent transmission of energy. The transducer device may further include a membrane connected to the transducer housing and disposed adjacent the emitting surface of the transducer element, and a cooling lumen for providing fluid to the membrane. A method of applying therapeutic ultrasound is also disclosed.

### BACKGROUND OF THE INVENTION

a. Field of the Invention

The instant invention is directed toward a multipurpose transducer device, including a finger-mounted or robot-mounted transducer device for therapeutic applications.

b. Background Art

In a normal heart, contraction and relaxation of the heart muscle (myocardium) takes place in an organized fashion as electrochemical signals pass sequentially through the myocardium from the sinoatrial (SA) node located in the right atrium to the atrialventricular (AV) node and then along a well defined route which includes the His-Purkinje system into the left and right ventricles. Atrial fibrillation results from disorganized electrical activity in the heart muscle, or myocardium. The surgical maze procedure has been developed for treating atrial fibrillation and involves the creation of a series of surgical incisions through the atrial myocardium in a preselected pattern so as to create conductive corridors of viable tissue bounded by scar tissue. As an alternative to the surgical incisions used in the maze procedure, an increasingly common medical procedure for the treatment of certain types of cardiac arrhythmia and atrial arrhythmia involves the ablation of tissue in the heart to cut off the path for stray or improper electrical signals.

Ablation may be performed either from within the chambers of the heart (endocardial ablation) using endovascular devices (e.g., catheters) introduced through arteries or veins, or from outside the heart (epicardial ablation) using devices introduced into the chest. The ablation devices are used to create elongated transmural lesions—that is, lesions extending through a sufficient thickness of the myocardium to block electrical conduction—which form the boundaries of the conductive corridors in the atrial myocardium. The catheter ablation devices create lesions at particular points in the cardiac tissue by physical contact of the cardiac tissue with the ablation element and the application of RF energy. Frequently the points are strung together to form elongated blocking lesions; however, this is quite difficult to do using the drag and burn approach with an endoluminal catheter. Ultrasound ablators have the advantage that they do not necessarily need to touch the tissue directly; they only need a water or tissue path free of air or gas between the ablator and the target. Frequently the water standoff is a saline-filled membrane.

One challenge in obtaining an adequate ablation lesion is the constant movement of the heart, especially when there is an erratic or irregular heart beat. Another difficulty in obtaining an adequate ablation lesion is retaining sufficient and uniform contact with the cardiac tissue across the entire length of the ablation element surface. Without sufficiently continuous and uniform contact, the associated ablation lesions may not be adequate. This problem is most severe with catheters.

In performing the maze procedure and its variants, whether using ablation or surgical incisions, it is generally considered most efficacious to include a transmural incision or lesion that isolates the pulmonary veins from the surrounding myocardium. The pulmonary veins connect the lungs to the left atrium of the heart, and join the left atrial wall on the posterior side of the heart. This location may create difficulties for endocardial ablation devices. The elongated and flexible catheter-based endovascular ablation devices are difficult to manipulate into the geometries required for forming pulmonary lesions and to maintain in such positions against the wall of a beating heart. This procedure is time-consuming and may result in lesions which do not completely encircle the pulmonary veins or which contain gaps or discontinuities.

An epicardial ablation device may be used to create continuous, linear lesions during cardiac ablation. The device may comprise a plurality of ablation cells connected together by a hinge wire. The hinge wire may be provided to connect the cells together so that they are configured to form a substantially complete compliant ring for generally encircling the cardiac tissue at the time of ablation. A degree of device shape adjustment should take place as the heart is not round. Each cell may comprise an ablation element, as well as a cell carrier for retaining the ablation element. The device may be positioned securely around a patient's atrium while the ablation elements apply energy (e.g., HIFU energy) to the targeted tissue. The term securely means non-sliding and non-slipping on the heart unless the device is specifically designed to slide on a progressive track in a controlled manner.

In procedures using such an epicardial ablation device, as well as other procedures and/or surgeries, the ability to easily and/or sufficiently access the tissue to be treated is of great significance. In some cases, procedures may be modified or avoided altogether because of the inability to easily and/or sufficiently access the tissue that has been targeted for treatment. While a large assortment of tools (e.g., surgical tools) have been designed with pliable or formable shapes in order to try to ameliorate this situation, there remain procedures and surgeries in which the available tools are not sufficient and/or preferred. In addition, the use of minimally-invasive surgeries (MIS) is increasing, which means that smaller incisions provide for even less ability to navigate tools and instruments.

Thus there remains a need for a device for procedures and/or surgeries that can be more easily manipulated and may support a minimally-invasive procedure and/or surgery.

### BRIEF SUMMARY OF THE INVENTION

It is desirable to provide a device for procedures and/or surgeries that may be more easily manipulated than currently available tools. For example, a finger-mounted tool (e.g., a tool mounted on a surgeon's gloved or ungloved fingers) may be one of the most easily manipulated instruments that are available. A finger-mounted tool may be utilized to treat tissues which may be readily reached with the fingers, as opposed to handled ablation devices or other tools and/or instruments. Further, the finger could also be a robot's “finger” or articulator.

A transducer device for therapeutic applications is disclosed. The transducer device may include a mounting body configured for mounting the device to a finger of an operator of the device or to a robot's articulator. The transducer device may also include a transducer housing connected to a mounting body that defines a receiving portion. The transducer device may further include at least one transducer element disposed in the receiving portion that is/are configured for connection to an energy supply and configured to transmit ablating energy from an emitting surface. The transducer device may further include a gas reservoir disposed between the transducer element and the mounting body that is configured to prevent backwards transmission of energy away from tissue. The transducer device may further include a membrane connected to the transducer housing and disposed adjacent the emitting surface of the transducer element, and a fluid lumen for providing cooling and/or acoustic coupling fluid to the membrane. A method of applying therapeutic ultrasound is also disclosed.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 is a partial sectional view of a finger-mounted transducer device in accordance with a first embodiment of the invention.

FIG. 2 is a perspective view of a finger-mounted transducer device in accordance with a second embodiment of the invention.

FIG. 3 is a perspective view of a finger-mounted transducer device in accordance with a third embodiment of the invention.

### DETAILED DESCRIPTION OF THE INVENTION

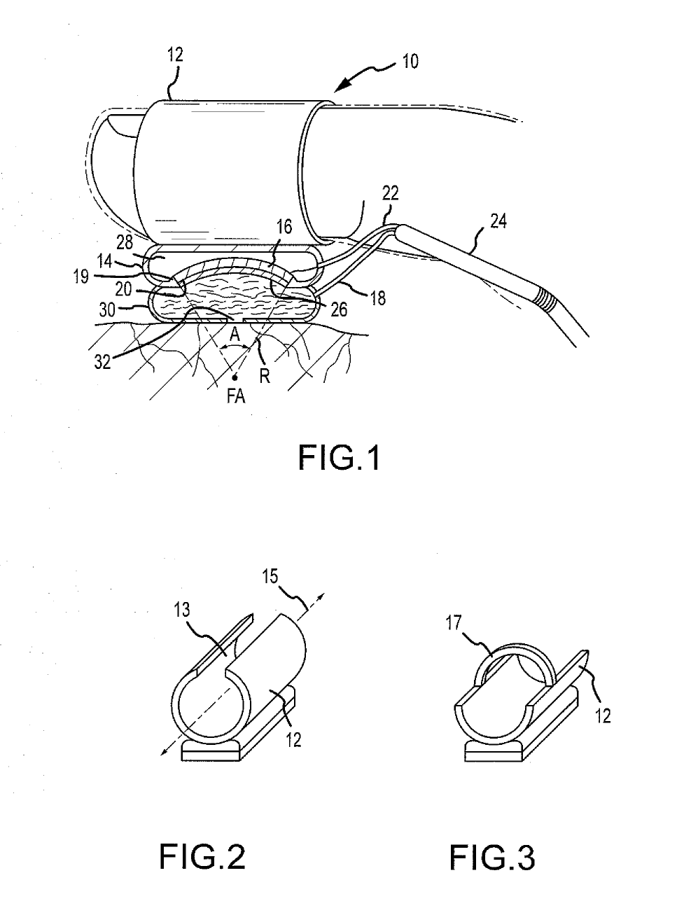

FIG. 1 illustrates a partial sectional view of a finger-mounted transducer device 10 in accordance with an embodiment of the invention. Device 10 may be configured for use in therapeutic applications. Device 10 may be mounted to a gloved finger as generally illustrated in the depicted embodiment. A surgical glove is illustrated in the figure in phantom. However, in other embodiments, device 10 may instead, for example, be mounted to a finger and then utilized under a glove with the ablative energy then passing through the glove in that case. While the device is described and illustrated as configured for connection (e.g., mounting) to a finger (e.g., a surgeon's finger), the device may also be configured for connection (e.g., mounting) to a robot finger-like appendage. The use of robots to perform procedures and/or surgeries is increasing, and the device can be configured to be utilized for various applications. The finger-mounted transducer device 10 may include a mounting body 12, a transducer housing 14, a transducer element 16, and a fluid cooling/coupling lumen 18.

Mounting body 12 may be configured for attaching or connecting device 10 to one or a plurality of fingers. A human finger may be replaced with a robotic finger or articulator with minimal change. In an embodiment, mounting body 12 may be configured for connection (e.g., mounting) to an index finger. In other embodiments, mounting body 12 may be configured for connection or mounting to an index finger and one or more other fingers. Mounting body 12 may, for example, comprise a cylindrical tube with a longitudinal axis as illustrated. Mounting body 12 may be configured to be slid onto a finger like an extended “ring.” In embodiments where the mounting body may be configured for mounting to an index finger and one or more other fingers, the mounting body may comprise two or more cylindrical tubes or generally tubular enclosures, each configured to be preferably slid onto a finger. Mounting body 12 may comprise a polymer or other suitably deformable (e.g., flexible or springy) material that will allow mounting body 12 to be slid onto or about a finger. Mounting body 12 may beneficially become deformed as the user's finger is inserted into the mounting body. The resistance of the mounting body to a slight deformation will likely provide sufficient pressure to the finger to firmly retain or hold the mounting body 12 on the finger. In another embodiment (see FIG. 2), mounting body 12 may form just a partial cylinder, so that the mounting body 12 may include a slit or opening 13 extending along the longitudinal axis 15 to allow for quick withdrawal of the finger from the mounting body. In another embodiment (FIG. 3), mounting body 12 may form a partial cylinder to receive the underside of a user's finger and then include a band 17 that may bridge the partial cylinder and be configured to retain mounting body 12 on a finger. Although these configurations are generally described in some detail, other configurations for connecting or mounting and retaining mounting body 12 on a finger may be utilized. Mounting body 12 may be configured with a streamlined shape and smooth edges so as to minimize irritation to the body cavity into which device 10 may be inserted. For the robotic application, the mounting body may be magnetically held to the robot finger as an alternative approach and/or a clip or fastener may be used.

Mounting body 12 may be provided in multiple sizes in accordance with an embodiment of the invention. Mounting body 12 may have a diameter that is configured to approximate the diameter of a range of user's fingers. Mounting body 12 may have an axial length that is about or less than a segment of a user's finger, e.g., the length of the tip of a finger beyond its distal joint, so as to not interfere with joint manipulation or movement. The mounting body may have an axial length that is configured to approximate the segment length of a range of user's fingers (e.g. an average length of about 20 mm and a range from about 15-50 mm). In other embodiments, mounting body 12 may have an axial length that is about or less than the length of a finger segment between adjacent joints (e.g., between the distal and middle joint and/or between the middle joint and proximal joint) so as to not interfere with joint manipulation or movement. In other embodiments, mounting body 12 may have an axial length that is greater than the length of a finger segment between joints, but may accommodate for comfortable manipulation.

Transducer housing 14 may be configured for permanent or detachable connection to mounting body 12. In accordance with an embodiment, at least one surface of transducer housing 14 may be configured for a snap-fit connection to mounting body 12. Although a snap-fit connection is mentioned, any other suitable connection could be used to connect transducer housing 14 to mounting body 12. For example, a clasp, clamp, fastener, adhering or adhesive member, magnet, vacuum, mating mechanical elements, or any combination thereof, may be utilized in other embodiments. In an embodiment, the finger may be oriented in the mounting body 12 such that the fingernail generally opposes the transducer housing 14 about the user's finger. Transducer housing 14 may include one or a plurality of walls that define a receiving portion configured to receive at least a portion of transducer element 16. Optionally, the transducer housing 14 may have curved portions 19 (e.g., lips) on both sides of the receiving portion that generally conform to the curvature of the transducer element 16.

Transducer element 16 may be configured to transmit and/or apply energy to target tissue. Transducer element 16 may include an emitting surface 20 from which the transducer element 18 transmits energy. A controller (not shown) may be provided to control delivery of energy. Transducer element 16 may comprise an ultrasonic transducer. In other embodiments, transducer element 16 may include a radiofrequency (RF) electrode, microwave transmitter, cryogenic element, laser, optical heating element, resistive heated element, hot fluid, or any of other known types of elements suitable for forming transmural lesions. In an embodiment where transducer element 16 is configured to transmit and/or apply HIFU energy, the controller may be comparatively simpler and be coupled to only a single power supply. The controller may control the delivery of energy from the energy source or power supply. A line or cable 22 may extend from the energy source to the transducer element 16 and connect to transducer element 16. The line or cable 22 may be provided to supply energy (e.g., electrical energy). The line or cable 22 may extend through an umbilical 24. In an embodiment, umbilical 24 may be connected to the transducer housing 14 (e.g., through a formation or protrusion for receiving the line or cable), and in other embodiments, may be configured to form a seal with transducer housing 14. Umbilical 24 may be routed up the finger and may be configured to hang free in an embodiment or, in other embodiments, may be configured for connection to the finger. In all cases, appropriate strain-relief could be provided where the cables or leads enter the transducer.

Transducer element 16 may comprise a piezoceramic element. Transducer element 16 may have a length and/or width and/or diameter of about 1.5 cm to about 2.5 cm. These dimensions are provided for illustrative purposes only. The dimensions, size, and shape of the finger-mounted transducer may vary in order to be able to support a selection of high intensity focused ultrasound (HIFU) lesion shapes via selection of the transducer element. In addition to the transducer geometry being utilized to form lesions, the transducer element may also be subjected to physical motion relative to the tissue to be targeted to assist in formation of lesions of desired size, shape, and location. Thus, the scope of the present invention encompasses any feature incorporated with such a physically scanned transducer which allows for tracking control or monitoring of such motion or articulation.

For some applications, transducer element 16 may be curved to physically focus its transmitted energy. The focus may be fixed mechanically in that manner by the shape of the transducer element 16. Acoustic designs for transducer element 16 may include a variety of fixed focus transducers, such as spherical and cylindrical transducer or lensed transducers of any type. The focus may be either a line-focus or point-focus. For example, transducer element 16 may be generally cylindrical in shape to focus to a line or may be generally spherical in shape to focus to a point. In the line-focus embodiment, the transducer element 16 may be configured for the line to be parallel or orthogonal to the axis of the finger. In other embodiments, the transducer element 16 may be pointed in different directions relative to the axis of the finger or plurality of fingers. Phased array electronic focusing and/or steering may also be utilized in accordance with the invention. In any of these approaches, the transducer may also be used for imaging or at least for performing lesion feedback. In an embodiment, a combined co-integrated imaging and HIFU transducer may be utilized, although this typically compromises both functions. The way to get around compromising both functions is to utilize physically separate imaging and HIFU transducers.

An acoustic matching layer 26 may be bonded or otherwise acoustically coupled to a concave side of transducer element 16. Layer 26 may comprise aluminum or any other suitable material. Layer 26 may have the same radius of curvature as transducer element 16 so that layer 26 mates with transducer element 16. Layer 26 may be attached to the curved lips of the transducer housing 14 with an epoxy. Although the focused ultrasonic energy may be produced with the curved transducer and layer, it may also be produced with a wide variety of other suitable structures. For example, acoustic lensing may be used to provide focused ultrasound. The acoustic lens may even be used with a flat piezoceramic element and matching layer. Finally, some transducers combine electronic steering or phasing and mechanical focusing or defocusing (e.g., a curvilinear imaging array).

Transducer element 16 may transmit focused ultrasound in at least one dimension. An advantage of using focused ultrasound is that the energy can be concentrated within the tissue. Another advantage of using focused ultrasound is that the energy diverges after reaching the focus, which can reduce the possibility of damaging tissue beyond the target tissue as compared to collimated ultrasonic energy. Although the ultrasound energy is emitted directly toward the tissue in an embodiment, the ultrasound energy may also be reflected off a surface and directed toward the tissue in other embodiments. Such a reflective surface could be flat in one embodiment or could be curved in another embodiment to focus the energy physically.

The focused ultrasound may, for example, have a focal length of about 2 to 20 mm. Transducer element 16 can have a radius of curvature R consistent with the desired focal lengths for a substantially point focus. The focused ultrasound may form an angle of 10 to 170 degrees as defined relative to a focal axis FA. The transducer element 16 may form an angle A with the focus within the desired angle ranges. A preferred angle range is about 60-90 degrees because this helps ensure good spot heating, as well as minimal downstream beyond-focus damages. The focused ultrasound will typically emit 90%-99% of the energy within the focal lengths and angles described above.

The finger or robot mounted transducer may further include a means for varying the energy deposition versus depth via operational frequency manipulation. The focal length may be adjusted by changing the distance from the emitting surface 20 of the transducer element 16 to the tissue to be targeted. To do this, the device 10 may be moved closer to and farther away from the target tissue with an adjustably inflatable membrane (for example, as described below) which may also beneficially generally conform to the shape to fill the gap between the transducer element 16 and the targeted tissue. The focus may be adjusted through the use of electronic timing adjustments to move the transducer element in and out in the case of the transducer having phased subelements such as a phased array. Transducer element 16 may be operated while varying one or more operational parameters, such as frequency, power, ablating time, and/or location of the focal axis relative to the targeted tissue. A supporting console (not shown) may be operated by the user that allows for adjustment of power supply, frequency, activation time, and any other characteristics. In an embodiment, the console may be operated utilizing a foot switch, a hand switch, a voice-activated switch, or other techniques known to those in the field.

In an embodiment, transducer element 16 may be operated at a frequency of about 4-6 MHz. Transducer element 16 may be operated at a power of about 80-140 watts, and in short bursts. For example, and without limitation, transducer element 16 may be operated for about 0.01 to 1.0 seconds. Transducer element 16 may then be inactive for about 2-90 seconds. Treatment at this frequency in relatively short bursts will produce localized heating at the focus. Energy may not be absorbed as quickly in tissue at this frequency as compared to higher frequencies so that heating at the focus is less affected by absorption in the tissue. In some embodiments, transducer element 16 may be operated for longer periods of time, for example and without limitation, about 1-4 seconds, in order to distribute more ultrasound energy between the focus and the near surface. Transducer element 16 may be operated at a power of about 20-60 watts. Transducer element 16 may be inactive for about 3-10 seconds. In other embodiments, transducer element 16 may be activated at higher frequencies to heat and ablate the near surface. For example, transducer element 16 may be activated at a frequency of about 6-20 MHz. Transducer element 16 may be operated at lower power in this embodiment, since ultrasound may be rapidly absorbed by the tissue at these frequencies so that the near surface may be heated quickly. There is a natural tendency for lesions to build back toward the transducer face during long ablations.

Transducer element 16 may be operated with the temperature near the surface of the tissue being about 43° C. to about 60° C. In some embodiments, the temperature near the surface of the tissue may go up to about 100° C. In general, when trying to ablate near-surface or shallow tissues, there is a benefit in permitting the transducer face temperature to rise since an additional thermal conduction mechanism of lesion forming at or near the surface may be attained in addition to the acoustic mechanism. An energy controller (not shown) may utilize feedback, such as temperature-based feedback or transducer driving electrical impedance, to actively control the amount of energy and to sense the degree of acoustic coupling. For example and without limitation, one or more temperature sensors on a finger or robot mounted transducer device 10 may be coupled to the controller. Ablation at the transducer element 16 may be controlled based on temperature measured at the temperature sensors. For example, the controller may be configured to maintain a near surface temperature (e.g., between about 43° C. and 100° C. degrees). The temperature may be adjusted, for instance, by changing the fluid flow rate and temperature and/or the power delivered to transducer element 16. Lesion feedback may also be obtained by analyzing a pulse-echo sequence passed into the formed lesion.

Device 10 may also include a plurality of ultrasonic transducers. Each of the ultrasonic transducers may have varying characteristics. For example, each of the transducer elements 16 of the plurality of ultrasonic transducers may provide ultrasound having different focal lengths (i.e., different depth focus) and/or be intended to operate at different frequencies or power. Device 10 can be configured for interchangeability of the transducer elements 16. For example and without limitation, the ultrasonic transducers could be interchanged during a procedure and/or surgery. In this manner, the operator may select the appropriate transducer element 16 to ablate a particular tissue structure and/or for another procedure or surgery. For example and without limitation, it may be desirable to select a transducer element with a small focal length and/or low power when ablating thin tissue. The scope of the present invention encompasses mounting of various size, shape or number of abutted transducers to obtain a desired lesion size and/or shape.

Device 10 may also utilize a plurality of transducer elements 16 that are oriented to focus or concentrate ultrasonic energy within preferred angle ranges and radius of curvature described herein. For example, a multi-element phased array may be utilized. Accordingly, the focused energy may be produced in a number of different ways.

Further, transducer element 16 will preferably be air-backed. By “air-backed” it is meant in the art and herein that the transducer is backed with one of air, a gas, a vacuum, or an air-filled porous or permeable material. A vacuum may not be preferred for some embodiments due to the associated expense. All of these materials are highly reflective to backwards going acoustic waves. For example, transducer element may be positioned on transducer housing 14, so that a gas (e.g., air) reservoir 28 may be disposed adjacent the transducer element (e.g., on a surface opposite to the emitting surface 20 of the transducer element 16). The use of air or another gas (or fluid) behind transducer element 16 will prevent ultrasound from going in the direction opposite to the direction of emitting surface 20. Accordingly, the energy is primarily directed from emitting surface 20 at the tissue. An air-backed configuration may not easily be used in connection with ultrasonic imaging probes. An ultrasonic imaging probe generally involves the use of a lossy backing material (e.g., a polymeric backing material to cause the dissipation of energy) to absorb the transmitted short pulses and stop the vibration associated with the transmitted short pulses. The lossy backing material is commonly incompatible with the power requirements necessary for therapeutic ultrasonic devices because such lossy backing material may overheat. On the other hand, an air-backed transducer may be used in connection with a therapeutic ultrasonic device because a therapeutic ultrasonic device utilizes a continuous wave for a longer period of time (and does not require a lossy backing material). In addition to being compatible with a therapeutic ultrasonic device, the gas reservoir 28 may also serve to minimize the heat removal requirements because it thermally insulates the transducer from the mounting body 12.

Even with the minimized heat removal requirements due to the use of a gas-backed (e.g., air-backed) transducer element 16, a means of cooling transducer element 16 may still be necessary. A HIFU transducer has the potential to burn a finger to which it has been mounted via conducted heat. Further, a thermally insulated transducer will boil its front-side liquid very quickly at high powers. A membrane 30 may be disposed in front of the emitting surface 20 of transducer element 16. Membrane 30 may be connected to transducer housing 14. In an embodiment, membrane 30 may be separately and fluidally sealed with transducer housing 14 with the exception of any needed input and output ports or orifices. The membrane 30 may be filled with a fluid or gel and may be provided to help transmit the energy from the transducer element 16 to the tissue to be targeted at low loss. Membrane 30 may be flexible and complaint in order to be able to conform to the required shape to fill a gap between transducer element 16 and the tissue to be treated. In an embodiment, membrane 30 may comprise a thin urethane or polyester. However, membrane 30 may comprise other suitable, flexible materials in other embodiments. Membrane 30 may also be permeable to water as opposed to having one or more laser-drilled or punched water orifices in its face to assure some flow and/or tissue irrigation/wetting. Membrane 30 can also be provided to vary the distance between the transducer element 16 and the tissue. For example, when ablating thick tissue, the membrane 30 may be fluidally deflated so that the transducer element 16 is closer to the target tissue. When ablating thin tissue, membrane 30 may be fluidally inflated so that the transducer element 16 is moved further from the target tissue. Membrane 30 may also be inflated and deflated to move the focus relative to the tissue (e.g., to different depths).

Cooling and/or tissue-irrigating/wetting lumen 18 may be provided to manipulate fluid or gel to/from the tissue contact interface for acoustic coupling and/or to the membrane interior for cooling purposes. The fluid is preferably comprised primarily of saline or other water-based medium if cooling is being done as it is most easily flowed. In other embodiments, the fluid may comprise any number of other fluids that may be used for cooling/coupling in the intended environment. The fluid should be a conductive fluid to allow conduction or passage of energy from the transducer element to the tissue. This usually means the fluid has low attenuation or low losses such that it is not itself directly heated by attenuating ablation energy. The source of fluid may include a saline-bag that provides a gravity feed and is coupled to the device 10 with a standard connection such as a standard luer connection. The fluid pressure will cause a net flow into and out of the liquid filled membrane. That outflow may include outflowed water which simply cools the transducer and/or surface tissue, and/or fluid which is emitted or leaked into the membrane/tissue interface to assure good wetted acoustic coupling. The membrane may have a positive displacement flow such that it cannot easily be collapsed.

Laser drilled hole(s) 32 to allow for flow or ingress of the cooling fluid (e.g., saline) between the outer surface of the membrane 30 and the tissue to be ablated are illustrated. If this interface were to dry out, such as with heating, any air cavity formed in the path of the ultrasound would cause ultrasound to bounce back toward emitting surface 20, so that the cooling fluid flowing between the outer surface of membrane 30 and the targeted tissue avoids such formation of an air cavity or film. Fluid passed into the membrane, particularly if it is saline, is most easily dumped into the body cavity and aspirated. Often this also provides an immersed environment for the transducer which can provide yet more cooling and help assure there is no intervening air films.

In an embodiment, the finger-mounted or robot-mounted transducer device 10 may be configured for co-integrated imaging. For example, and without limitation, an existing laptop ultrasound system (such as the Sonoscan™ offered by Sonosite Inc. of Bothell, Wash.) may be utilized to support such imaging. The finger-mounted transducer device 10 may incorporate ultrasonic imaging by a combined HIFU/imaging transducer or by independent HIFU and imaging transducers. In an embodiment where the finger-mounted transducer device 10 is configured for co-integrated imaging, the ultrasonic transducer element 16 may be non-disposable, but may include disposable HIFU inserts/elements or disposable coupling elements. In another embodiment where the finger-mounted or robot-mounted transducer device 10 is configured for co-integrated imaging, the transducer element 16 may be non-disposable, but may include a disposable standoff/coolant spacer. In an embodiment, the spacer may comprise or include an acoustically transmissive standoff, like a saline filled membrane. In other embodiments, the spacer can be non-flowing, such as a gel standoff, and provide a more convenient working distance to tissue. These embodiments may require the use of an added small box to drive the HIFU unit. The finger-mounted or robot-mounted transducer device 10 may be used for obtaining measurements, e.g., temperature, tissue thickness, thickness of fat or muscle layers, and blood velocity data. The finger-mounted transducer device 10, utilizing ultrasound, may also be uses to assess the adequacy of contact between the device 10 and the tissue to be ablated. In the embodiment of the finger-mounted or robot-mounted transducer device 10 configured for co-integrated imaging, the device 10 may be configured for the HIFU transducer element 16 to be mounted on a first finger and for the ultrasonic imaging transducer element to be mounted on a second finger. In an embodiment, the HIFU transducer element may be opposite to or facing the ultrasonic imaging transducer through the targeted tissue in use. Thus, the performance tradeoff of a combined HIFU and imaging transducer can be avoided by closely spacing the two different type transducers. This technique is known by persons of ordinary skill in the art.

Although the device 10 may be utilized in connection with ultrasonic imaging and/or may be utilized in connection with a three dimensional, anatomical mapping and localization system (e.g., the NavX™ system provided by St. Jude Medical) to provide information and data regarding the location of the device 10 and/or tissues to be ablated, in other embodiments the device 10 may be operated unguided and/or without imaging. The device 10 may be utilized absent direct visualization of the device 10 (e.g., out of operator sight). Such could easily be the case if the device 10 were mounted on a robotic arm or carried by a miniature crawling robot inside the body. The scope of the present invention encompasses the use of an external imaging device such as magnetic resonance imaging (MRI), fluoroscopy, CATSCAN, or even externally coupled ultrasound imaging probes, as long as there are no intervening air pockets between the probe and the ablator.

In some embodiments, the finger-mounted or robot-mounted transducer device 10 may include a Doppler generator/detector. The Doppler generator/detector may be mounted in the device 10 for directing, detecting, and transmitting signals representative of blood flow velocity through a vessel contacted by the undersurface of the device 10. The Doppler generator/detector may use flow sensing to locate bleeding. Accordingly, a practitioner or surgeon may be able to collect anatomic and hemodynamic information of vascular segments through touching the surface of interest with his or her fingertip. The Doppler generator/detector may be oriented so that it faces toward an imaging plane and in a direction along, or at angle to (e.g., at an angle of 45 degrees), the flow of blood through a vessel contacted by the device 10. Leads may connect the device 10 to suitable instrumentation for generating ultrasonic pulses and for detecting the echoes and determining flow velocity as represented by the Doppler shift. Such instrumentation is well known and commercially available. However, the use of imaging, such as ultrasound imaging, is preferred for some embodiments because it provides readily at hand color-flow Doppler imaging, directed-Doppler, as well as other useful modalities for assessing lesions such as tissue-elasticity imaging.

In some embodiments, device 10 may include additional instrumentation to assure a minimum cooling fluid flow even if excessive finger pressure is applied. For example, the device 10 may include a flow meter or pressure sensor. The flow meter can permit the operator to see the fluid flowing on the far side of the device. In another example, the device 10 may include a positive displacement pump, instead of a gravity feed. Even if the membrane is nearly collapsed, the positive displacement pump will protectively force fluid past the transducer. In some embodiments, both a pump (i.e., force flow in place of gravity feed) and a flow meter may be utilized.

The finger-mounted or robot-mounted transducer may also integrate or be used with thermistors or thermocouples, with pacing or sensing electrodes, with a video camera chip, or with a spatial location and orientation-tracking mechanism, such that it can be traced in 3D. In some embodiments, the transducer may incorporate vacuum-suction for purposes of target-tissue or even finger-fixation in cases if desired.

Transducer device 10 may be configured for potential disposability. In an embodiment, the finger-mounted or robot-mounted transducer may be configured to be reuseable, partly disposable, or fully disposable. For example and without limitation, the finger-mounted or robot-mounted transducer device 10 may include a disposable transducer element 16 or a disposable mounting body 12. In another embodiment, the finger-mounted transducer device 10 may include a disposable sheath (not shown). For example, the transducer element 16 may be encased in a sheath (e.g., membrane). After use, the sheath may be removed from the transducer element 16 and thrown away. The transducer element 16 itself may then be put in a chemical dip or a wet sterilant to be reused in a new sheath. The non-disposable device 10 may thus utilize various replaceable, disposable transducer-related elements in one embodiment, or a plurality of the same disposable transducer-related elements in another embodiment. In another embodiment, the finger-mounted transducer device 10 may be disposable in its entirety.

In addition to ablation, the finger-mounted transducer device 10 may have numerous applications, including the following, for example and without limitation: (a) surgical wound closure; (b) catheterization wound closure; (c) lesion formation supporting the maze procedure; (d) coagulation to reduce bleeding during later surgical incision; (e) removal of diseased liver lobes; (f) cosmetic fat reduction; (g) trauma/battlefield care; (h) cancer treatment; (i) formation of lesions endocardially using fingers and pursestring sutures, if necessary or preferable; (j) stoppage of uncontrolled bleeding; (k) brain surgery; (l) breast cancer surgery; (m) skin wrinkle reduction, and/or (n) surgical bleeding minimization as by pre-cauterization of regions around incisions.

While the preferred ablator herein is a piezoceramic based ultrasonic transducer with mechanical focusing, an ablation laser may also be employed to focus energy at depth in an embodiment. In that case, the delivery fiber may be run along the user's (or robot's) arm and either have a sharp bend in the fiber or use a mirror to redirect the laser energy toward the tissue at the user's finger (unless it is instead directed off the user's fingertip). In the case of the laser ablator, the flowed water, if used, will assure that boiling does not happen at the tissue surface or laser fiber output window. Microwave may be moderately focused if used as the ablator in an embodiment of the invention. RF and cryoablation energies are unfocused, but may be used in accordance with embodiments of the invention.

In the case of robotic application, mounting body 12 may mate with the robot's appendage. This task allows for more variety in the mounting body design. In a preferred robotic approach, the cooling and/or coupling fluid and lumen may be utilized. The robotic system would likely be integrated with the HIFU device and carry both the robot's typical video camera, as well as a desirable ultrasound imaging array to guide and assess HIFU lesioning. For spatial positioning, the robot may have its own such system or may rely on the HIFU probe's spatial location sensor.

For the ablative treatment of a cardiac rhythm disorder, such as atrial fibrillation (AF), we anticipate the robotic use of an ablator in accordance with an embodiment of the invention combined with an electrophysiology mapping device such as the St. Jude Medical EnSite™ mapping device or an analogous mapping product available from Biosense Webster.

Although several embodiments of this invention have been described above with a certain degree of particularity, those skilled in the art could make numerous alterations to the disclosed embodiments without departing from the spirit or scope of this invention. All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A transducer device for therapeutic applications, comprising:
a mounting body configured for connecting or mounting said device to at least one human finger of an operator or to a robotic articulator of a robot;
a transducer having at least one energy emitting element configured for connection to an energy supply and for transmitting therapeutic energy from an emitting surface, said at least one energy emitting element connected to or mounted upon said mounting body; and
a lumen for providing fluid to said at least one energy emitting element.

2. The device of claim 1, wherein said transducer is an acoustic or ultrasonic transducer.

3. The device of claim 2, wherein said acoustic transducer is acoustically backed using an air-like, gas-like, or vacuum-like region.

4. The device of claim 1, wherein said mounting body comprises at least a partially formed generally cylindrical tube or passage into or onto which said finger or said articulator rests or is inserted.

5. The device of claim 1, wherein said mounting body comprises a flexible, malleable, or elastic polymer or metal.

6. The device of claim 1, wherein said mounting body is configured to have an axial length that is about equal to or less than the length of a finger segment.

7. The device of claim 1, further comprising a transducer housing connected to said mounting body with a snap, clasp, clamp, fastener, adhering or adhesive member, magnet, vacuum, mating mechanical element, or any combination thereof.

8. The device of claim 1, wherein said at least one energy emitting element comprises an ultrasonic transducer, laser, cryogenic element, RF electrode, microwave emitter, an optical energy heating source or an electrically resistive heating source.

9. The device of claim 1, wherein the maximum lateral dimension of a transducer energy emitting face of said at least one energy emitting element is about 2.5 centimeters or less.

10. The device of claim 1, wherein said at least one energy emitting element has a shaped profile to direct energy; multiple elements which steer energy by the utilization of timed phase delays; an acoustic lens or acoustic reflector for directing energy; an optical lens or optical reflector for directing energy; a tissue-contacting or tissue-facing face which serves as an electrode; imaging capability; the ability to assess a tissue targeted for therapy; or a physical standoff from tissue which is substantially transparent to some therapeutic energy; or combinations of one or more of the foregoing.

11. The device of claim 1, further comprising a temperature sensor coupled to said device, wherein said temperature sensor provides feedback which is employed for a safety or therapy management or control reason.

12. The device of claim 1, further comprising a membrane disposed between said transducer and a tissue portion.

13. The device of claim 12, wherein said membrane is flexible.

14. The device of claim 12, wherein said membrane includes at least one aperture for facilitating flow or leakage of coolant into an interface between said membrane and said tissue portion.

15. The device of claim 1, wherein said transducer is a therapy transducer, and further comprising an imaging transducer which is co-mounted with said therapy transducer.

16. The device of claim 1, wherein said transducer is a therapy transducer integrated with an imaging transducer to form a single integrated transducer.

17. The device of claim 1, wherein said transducer performs a Doppler flow function.

18. The device of claim 1, wherein said lumen provides fluid to said at least one energy emitting element to cool said at least one energy emitting element, cool tissue receiving said therapeutic energy, control temperature, provide energy coupling, provide transducer manipulation, or combinations thereof.

19. The device of claim 1, wherein at least one of the transducer, the mounting body, or the lumen is disposable.

20. A method of applying therapeutic ultrasound to tissue, comprising:
mounting a transducer device to an operator's finger or to a robotic articulator of a robot, wherein said transducer device includes:
a transducer having at least one energy emitting element configured for connection to an energy supply and configured to transmit therapeutic energy from an emitting surface; and
a gas reservoir disposed between said transducer element and said finger to prevent transmission of said energy; and
supplying a cooling fluid to at least one energy emitting element.

21. The method of claim 20, further comprising varying the focal length of said energy.

22. The method of claim 20, further comprising varying the frequency of said energy.

23. The method of claim 20, comprising operating said transducer element at a frequency of about 4-6 MHz.

24. The method of claim 20, comprising operating said transducer element continuously for a period of about 0.1 to 1.0 seconds.

25. The method of claim 18, further comprising inflating or deflating a membrane for varying the distance between said transducer and tissue, wherein at least a portion of said membrane is connected to said device and disposed between said transducer and said tissue.

26. The method of claim 20, wherein said finger of said operator is gloved.

27. The method of claim 20, further comprising placing a glove over said transducer device.

28. A transducer device for therapeutic applications, comprising:
means for mounting said device to a finger of an operator of said device;
means for transmitting energy from said device;
means for preventing transmission of said energy toward said finger; and
means for providing cooling fluid to said device.
